SEQUENCE LISTING (96 sequences)

SEQ ID NO: 1 - protein, 12 aa, Artificial
  Binding peptide recognized by antibody
NNSYLDEEGTWL

SEQ ID NO: 2 - protein, 12 aa, Artificial
  Binding peptide recognized by antibody
FPTHNPTSPLDM

SEQ ID NO: 3 - protein, 12 aa, Artificial
  Binding peptide recognized by antibody
DLNDPPFFTSVS

SEQ ID NO: 4 - protein, 12 aa, Artificial
  Binding peptide recognized by antibody
TTHWDHPFYDNT

SEQ ID NO: 5 - protein, 12 aa, Artificial
  Binding peptide recognized by antibody
AWHPSSPLDYGF

SEQ ID NO: 6 - protein, 12 aa, Artificial
  Binding peptide recognized by antibody
ANSVVYKPSSPL

SEQ ID NO: 7 - protein, 12 aa, Artificial
  Binding peptide recognized by antibody
TSYNTDHPFHYP

SEQ ID NO: 8 - protein, 12 aa, Artificial
  Binding peptide recognized by antibody
SPSAPSSPLDSM

SEQ ID NO: 9 - protein, 12 aa, Artificial
  Binding peptide recognized by antibody
VRACVDCDQPFY

SEQ ID NO: 10 - protein, 12 aa, Artificial
  Binding peptide recognized by antibody
IAPSSFLDERTD

SEQ ID NO: 11 - protein, 12 aa, Artificial
  Binding peptide recognized by antibody
TRPHTDPPFWWA

SEQ ID NO: 12 - protein, 12 aa, Artificial
  Binding peptide recognized by antibody
EPDPPFYTHLTA

SEQ ID NO: 13 - protein, 12 aa, Artificial
  Binding peptide recognized by antibody
SQMKPSSHLDWD

SEQ ID NO: 14 - protein, 12 aa, Artificial
  Binding peptide recognized by antibody
HTLPQFLEWHKR

SEQ ID NO: 15 - protein, 12 aa, Artificial
  Binding peptide recognized by antibody
GQVVFQDWLLVR

SEQ ID NO: 16 - protein, 12 aa, Artificial
  Binding peptide recognized by antibody
HENANKIAPWVQ

SEQ ID NO: 17 - protein, 12 aa, Artificial
  Binding peptide recognized by antibody
QLKIAPWVIESP

SEQ ID NO: 18 - protein, 12 aa, Artificial
  Binding peptide recognized by antibody
TDTLDSFLGRSP

SEQ ID NO: 19 - protein, 12 aa, Artificial
  Binding peptide recognized by antibody
KPPRQVVAPWVM

SEQ ID NO: 20 - protein, 12 aa, Artificial
  Binding peptide recognized by antibody
IAQTLPAFLATR

SEQ ID NO: 21 - protein, 12 aa, Artificial
  Binding peptide recognized by antibody
TETLHEFLDGRK

SEQ ID NO: 22 - protein, 12 aa, Artificial
  Binding peptide recognized by antibody
KHLEHPTYHLWH

SEQ ID NO: 23 - protein, 12 aa, Artificial
  Binding peptide recognized by antibody
SLNTIAPWILTA

SEQ ID NO: 24 - protein, 12 aa, Artificial
  Binding peptide recognized by antibody
KATTHNIAWWVA

SEQ ID NO: 25 - protein, 12 aa, Artificial
  Binding peptide recognized by antibody
HDNINMAWWVEW

SEQ ID NO: 26 - protein, 12 aa, Artificial
  Binding peptide recognized by antibody
EWYVSTSLLPLP

SEQ ID NO: 27 - protein, 12 aa, Artificial
  Binding peptide recognized by antibody
HIYYISESLLPM

SEQ ID NO: 28 - protein, 12 aa, Artificial
  Binding peptide recognized by antibody
HVEYEIIEQMIY

SEQ ID NO: 29 - protein, 12 aa, Artificial
  Binding peptide recognized by antibody
SPFFYTLTRVPM

SEQ ID NO: 30 - protein, 12 aa, Artificial
  Binding peptide recognized by antibody
SSYLYTTQFVPL

SEQ ID NO: 31 - protein, 12 aa, Artificial
  Binding peptide recognized by antibody
TGWMISTTLIPT

SEQ ID NO: 32 - protein, 12 aa, Artificial
  Binding peptide recognized by antibody
YKYSYTTELILI

SEQ ID NO: 33 - protein, 12 aa, Artificial
  Binding peptide recognized by antibody
SYTYTTHLLPLA

SEQ ID NO: 34 - protein, 12 aa, Artificial
  Binding peptide recognized by antibody
TMTQYYYSTTHY

SEQ ID NO: 35 - protein, 12 aa, Artificial
  Binding peptide recognized by antibody
QFWTITTTLVPH

SEQ ID NO: 36 - protein, 12 aa, Artificial
  Binding peptide recognized by antibody
GPKMDALSKAEN

SEQ ID NO: 37 - protein, 12 aa, Artificial
  Binding peptide recognized by antibody
GPRWLDALFMAE

SEQ ID NO: 38 - protein, 12 aa, Artificial
  Binding peptide recognized by antibody
HIGAHDSFETKH

SEQ ID NO: 39 - protein, 12 aa, Artificial
  Binding peptide recognized by antibody
NNSYLDEEGTWL

SEQ ID NO: 40 - protein, 12 aa, Artificial
  Binding peptide recognized by antibody
AMNTVVCYDEPC

SEQ ID NO: 41 - protein, 12 aa, Artificial
  Binding peptide recognized by antibody
TNSPLDELGTHP

SEQ ID NO: 42 - protein, 12 aa, Artificial
  Binding peptide recognized by antibody
STNSHEHWPMSP

SEQ ID NO: 43 - protein, 12 aa, Artificial
  Binding peptide recognized by antibody
NHFSSLGILTAA

SEQ ID NO: 44 - protein, 12 aa, Artificial
  Binding peptide recognized by antibody
IPQPSILTTPML

SEQ ID NO: 45 - protein, 12 aa, Artificial
  Binding peptide recognized by antibody
LRLPNTYENAKN

SEQ ID NO: 46 - protein, 12 aa, Artificial
  Binding peptide recognized by antibody
GPLSPAHLSQRN

SEQ ID NO: 47 - protein, 12 aa, Artificial
  Binding peptide recognized by antibody
FPLPFDPEMLPL

SEQ ID NO: 48 - protein, 12 aa, Artificial
  Binding peptide recognized by antibody
FPLPPEYSMKKD

SEQ ID NO: 49 - protein, 12 aa, Artificial
  Binding peptide recognized by antibody
FKIPEGNNVSVL

SEQ ID NO: 50 - protein, 12 aa, Artificial
  Binding peptide recognized by antibody
FRLPPHDSSDHH

SEQ ID NO: 51 - protein, 12 aa, Artificial
  Binding peptide recognized by antibody
FFLPHSATLPSH

SEQ ID NO: 52 - protein, 12 aa, Artificial
  Binding peptide recognized by antibody
FILPHRNGYIDT

SEQ ID NO: 53 - protein, 12 aa, Artificial
  Binding peptide recognized by antibody
LKLPNPDTAAPT

SEQ ID NO: 54 - protein, 12 aa, Artificial
  Binding peptide recognized by antibody
NNNMGNILLITP

SEQ ID NO: 55 - protein, 12 aa, Artificial
  Binding peptide recognized by antibody
VLLVPHHSHQYD

SEQ ID NO: 56 - protein, 12 aa, Artificial
  Binding peptide recognized by antibody
YLKESTHVLLAA

SEQ ID NO: 57 - protein, 12 aa, Artificial
  Binding peptide recognized by antibody
HDDWYISTSHTD

SEQ ID NO: 58 - protein, 12 aa, Artificial
  Binding peptide recognized by antibody
SYTYTTHLLPLA

SEQ ID NO: 59 - protein, 12 aa, Artificial
  Binding peptide recognized by antibody
EWYVYEKLFPLP

SEQ ID NO: 60 - protein, 12 aa, Artificial
  Binding peptide recognized by antibody
YKYSYTTELILI

SEQ ID NO: 61 - protein, 12 aa, Artificial
  Binding peptide recognized by antibody
HQYYYTSSLIYT

SEQ ID NO: 62 - protein, 12 aa, Artificial
  Binding peptide recognized by antibody
TAWHTTEELIML

SEQ ID NO: 63 - protein, 12 aa, Artificial
  Binding peptide recognized by antibody
NGYIFTTQLIWA

SEQ ID NO: 64 - protein, 12 aa, Artificial
  Binding peptide recognized by antibody
TVNPLLRLLSMG

SEQ ID NO: 65 - protein, 12 aa, Artificial
  Binding peptide recognized by antibody
NNPMLVLLSHGA

SEQ ID NO: 66 - protein, 12 aa, Artificial
  Binding peptide recognized by antibody
TVPQPQRLTQKS

SEQ ID NO: 67 - protein, 12 aa, Artificial
  Binding peptide recognized by antibody
AKYESLLRMLAA

SEQ ID NO: 68 - protein, 12 aa, Artificial
  Binding peptide recognized by antibody
GHESGYHISERA

SEQ ID NO: 69 - protein, 12 aa, Artificial
  Binding peptide recognized by antibody
GHEEHLWQILHF

SEQ ID NO: 70 - protein, 12 aa, Artificial
  Binding peptide recognized by antibody
GHEEQRNWSMEG

SEQ ID NO: 71 - protein, 12 aa, Artificial
  Binding peptide recognized by antibody
STYTDMLADLSA

SEQ ID NO: 72 - protein, 12 aa, Artificial
  Binding peptide recognized by antibody
SYESLLMKLARG

SEQ ID NO: 73 - protein, 12 aa, Artificial
  Binding peptide recognized by antibody
YRDSPGPRDKLL

SEQ ID NO: 74 - protein, 12 aa, Artificial
  Binding peptide recognized by antibody
GHEEREYQMMLQ

SEQ ID NO: 75 - protein, 12 aa, Artificial
  Binding peptide recognized by antibody
CLPYPLCRGTAT

SEQ ID NO: 76 - protein, 12 aa, Artificial
  Binding peptide recognized by antibody
TECLPYPLCLYN

SEQ ID NO: 77 - protein, 117 aa, Artificial
  Antibody heavy chain variable region
EVQLQQSGADLVKPGASVKLSCTASGFNIKDTYMHWMKQRPEQGLEWIGRIDPANGNSKYDPKFQGKATITADTSSNTAYLQLSSLTSEDTAVYFCTKSAGVPFAYWGQGTLVTVSA

SEQ ID NO: 78 - protein, 12 aa, Artificial
  Binding peptide recognized by antibody
NETWSEFWRLHN

SEQ ID NO: 79 - protein, 12 aa, Artificial
  Binding peptide recognized by antibody
TAQDDRIFHMMH

SEQ ID NO: 80 - protein, 12 aa, Artificial
  Binding peptide recognized by antibody
RGTQDMEYFAMH

SEQ ID NO: 81 - protein, 118 aa, Artificial
  Antibody heavy chain variable region
EVMLVESGGGLVKPGGSLKLSCAASEFTFSNYAMSWVRQTPEKRLEWVATISSGGSFTYYPDNLKGRFTVSRDNAKDTLYLQMSSLRSEDTAIYYCARQSLGYYFDSWGQGTTLTVSS

SEQ ID NO: 82 - protein, 12 aa, Artificial
  Binding peptide recognized by antibody
SVPWWTQSLLMS

SEQ ID NO: 83 - protein, 119 aa, Artificial
  Antibody heavy chain variable region
EVQLQQSGPVLVKPGASVKISCKTSGYTFTEYTIHWMRQSHGKSLEWIGGINPNKGNTNFNQKFKGKATLTVDKSSSTAYMELHSLPSEDSAVFYCARANWDVYAVDSWGQGTSVTVSS

SEQ ID NO: 84 - protein, 12 aa, Artificial
  Binding peptide recognized by antibody
DIWSTMEHNKYN

SEQ ID NO: 85 - protein, 12 aa, Artificial
  Binding peptide recognized by antibody
HINAIQYPLPHT

SEQ ID NO: 86 - protein, 119 aa, Artificial
  Antibody heavy chain variable region
QVQLQQPGAELVRPGASVKLSCKASGYSFASYWMNWVKQRPGQGLEWIGMIHPSDSGTSLDEKFKDKATLTVDKSSNTAYIQLNSPTSEDSAVYYCAREGYDPAWFAYWGQGTLVTVSA

SEQ ID NO: 87 - protein, 400 aa, Artificial [sequence omitted]

SEQ ID NO: 88 - protein, 112 aa, Artificial
  Antibody heavy chain variable region
DVQLQESGPGLVKPSQSLSLTCTVTGYSIINDYAWNWIRQFPGNKLEWMAYISYGGSIGYKPSLKSRISITRDTSKNQFFLQLNSVTTEDTATYYCARGGFYALDYWGQGTS

SEQ ID NO: 89 - protein, 118 aa, Artificial
  Antibody heavy chain variable region
EVQLQESGPSLVKPSQTLSLTCSVTGDSITSGSWNWIREFPGNKLEYMGYISYSGSTDYSPSLKSRISITRDTSKNQYYLQLNSVTTEDTATYYCATHYYGYLSLDYWGQGTSVTVSS

SEQ ID NO: 90 - protein, 114 aa, Artificial
  Antibody heavy chain variable region
EVQLVESGGGLVQPKGSLKLSCAASGFTFNTYAMSWVRQAPGKGLEWISRIRSKSNNYATYYVDSVKDRFTIYRDDSQSMLYLQMNNLKTEDTAIYYCVREGDWGQGTLVTVSA

SEQ ID NO: 91 - protein, 114 aa, Artificial
  Antibody heavy chain variable region
EVQLVESGGGLVQPKGSLKLSCAASGFTFSTSAMNWVRQAPGKGLEWISRIRSKTNNYATYYVDSVKDRFTIYRDDSQNMLYLQMNNLKTEDTAMYYCVREGDWGQGTLVTVSA

SEQ ID NO: 92 - protein, 122 aa, Artificial
  Antibody heavy chain variable region
QIQLVQSGPELKKPGETVEISCKASGYTFTDYSMHWVKQAPGKGLKWMGWINTETGEPTYADDFKGRFAFSLETSATTAYLQINNLKNEDTATYFCARGGYYGNYDYAMDYWGQGTSLTVSS

SEQ ID NO: 93 - protein, 15 aa, Artificial
  CYFRA protein
DVRADSERQNQEYQR

SEQ ID NO: 94 - protein, 15 aa, Artificial
  CYFRA protein
SERQNQEYQRLMDIK

SEQ ID NO: 95 - protein, 15 aa, Artificial
  CYFRA protein
QEYQRLMDIKSRLEQ

SEQ ID NO: 96 - protein, 15 aa, Artificial
  CYFRA protein
MKAALEDTLAETEAR

(19) United States
(12) Patent Application Publication
(10) Pub. No.: US 2014/0364326 A1
(43) Pub. Date: Dec. 11, 2014

(54) COMBINATORIAL BIOMARKERS FOR CLINICAL APPLICATIONS IN LUNG CANCER PATIENT MANAGEMENT

(71) Applicants: Mariana Guergova-Kuras, Chatillon (FR); Yann Kieffer, Maisons--Alfort (FR); Carole Malderez-Bloes, Saint-Michel-Sur-Orge (FR); Alexandra Kremeurt, Evry (FR); Laszlo Takacs, Bourg La Reine (FR)

(72) Inventors: Mariana Guergova-Kuras, Chatillon (FR); Yann Kieffer, Maisons--Alfort (FR); Carole Malderez-Bloes, Saint-Michel-Sur-Orge (FR); Alexandra Kremeurt, Evry (FR); Laszlo Takacs, Bourg La Reine (FR)

(73) Assignee: F. HOFFMANN-LA ROCHE SA, Basel (CH)

(21) Appl. No.: 13/981,725

(22) PCT Filed: Jan. 30, 2012
(86) PCT No.: PCT/EP2012/051463

§ 371 Date: Mar. 7, 2014

Related U.S. Application Data
(60) Provisional application No. 61/437,090, filed on Jan. 28, 2011.

(51) Int. Cl. G01N 33/574

(52) U.S. Cl. CPC G01N 33/57423
USPC 506/9; 506/18

(57) ABSTRACT

The present invention relates to compositions and methods for detecting, managing or monitoring cancer. The invention also relates to antibodies specific for cancer markers, compositions and chips containing the same, as well as their uses for cancer detection, managing, monitoring, imaging or treatment, as well as for drug development. The invention is particularly suited for detecting, managing or monitoring lung cancer in human subjects.

The present invention relates to compositions and methods for detecting, managing or monitoring cancer. The invention also relates to antibodies specific for cancer markers, compositions and chips containing the same, as well as their uses for cancer detection, managing, monitoring, imaging or treatment, as well as for drug development. The invention is particularly suited for detecting, managing or monitoring lung cancer in human subjects.

The present invention more specifically relates to the assessment of lung cancer using a combination of protein biomarkers. It discloses the use of CYFRA in combination with either one of the following proteins or their combinations: haptoglobin (HPT), alpha-1-antichymotrypsin (ACT), leucine-rich alpha-2 glycoprotein 1 (LRG-1), and complement factor 9 (C9). Measurement of anyone of these combinations may be used for the early detection or for the follow-up of patients having lung cancer, including NSCLC cancer, with high sensitivity and selectivity.

### BACKGROUND

Lung cancer is the most common cause of death from cancer and each year 1.4 million new cases are diagnosed worldwide. More than two-thirds of lung cancers are diagnosed at a late stage, when clinical symptoms appear. The overall survival rate after diagnosis ranges from 14% in the USA to 1.1% in some regions of Asia¹ and is currently very low due to the late diagnosis of the disease. Current diagnostics methods are based on imaging techniques and invasive procedures such as bronchoscopy or biopsy². The few known plasma biomarkers for lung cancer, such as carcinoembryionic antigen (CEA), squamous cell carcinoma antigen (SCC) and neuron-specific enolase (NSE) lack sufficient sensitivity and specificity³ to be used as early diagnostics tools. Ongoing efforts using high throughput discovery technologies are still struggling to provide reliable and easily accessible lung cancer biomarkers to enter the clinic^{4, 5}.

Global proteome analysis has been hampered by a variety of methodological problems including the fact that current mass spectrometry based proteome profiling technologies are in general far from covering the necessary dynamic range; are not adequately sensitive and lack sufficient reproducibility and throughput⁶. Polyclonal and monoclonal antibodies (mAbs) provide appropriately validated tools for the characterization and quantitative analysis of proteins but have primarily been targeted at a set of epitopes with low complexity^{7, 8}. Global, antibody proteomics approaches aim to generate libraries of antibodies to cover most or all individual proteins and their immunogenic epitops in any complex proteome. Recombinant phage, bacterial and viral display represent approaches to global antibody generation but have had limited success in resulting of libraries capable to detect complex proteomes with sufficient quality affinity reagents and coverage^{9 10} The Human Protein Atlas¹¹ and other large initiatives such as the NCI Clinical Proteomics Technology Initiative¹² are targeted at the generation of comprehensive libraries to the far more complex human proteome with both approaches using recombinant proteins as immunogens. The problem with recombinant proteins is that they do not represent the protein natural state, lack post-translational modifications and correct folding, therefore limiting the potential of the obtained libraries to profile natural proteomes.

### SUMMARY OF THE INVENTION

The present invention relates to the assessment or monitoring of lung cancer (“LC”) using a combination of protein biomarkers. It discloses the use of a CYFRA protein in combination with either one of the following proteins or their combinations: haptoglobin (HPT), alpha-1-antichymotrypsin (ACT), leucine-rich alpha-2 glycoprotein 1 (LRG-1), and complement factor 9 (C9), for detecting or monitoring lung cancer in human subjects. Surprisingly, the application shows that measurement of anyone of these combinations allows the early detection or the follow-up of patients having lung cancer, including NSCLC cancer, with remarkably high sensitivity and selectivity.

The invention also relates to kits or devices suitable for implementing the above methods.

The invention is particularly suited to detect or monitor lung cancer in human subjects, particularly Stage I and II, e.g., Stage Ia and IIa lung cancer.

An object of the invention relates to the use of CYFRA in combination with LRG-1 for the detection of LC.

Another object of the invention relates to the use of CYFRA in combination with ACT for the detection of LC.

Another object of the invention relates to the use of CYFRA in combination with C9 for the detection of LC.

Another object of the invention relates to the use of CYFRA in combination with HPT for the detection of LC.

Another object of the invention relates to the use of CYFRA in combination with at least LRG-1, ACT, HPT and C9 for the detection of LC.

Another object of the invention relates to the use of CYFRA in combination with LRG-1 for the diagnosis and/or management of LC.

Another object of the invention relates to the use of CYFRA in combination with ACT for the diagnosis and/or management of LC.

Another object of the invention relates to the use of CYFRA in combination with C9 for the diagnosis and/or management of LC.

Another object of the invention relates to the use of CYFRA in combination with HPT for the diagnosis and/or management of LC.

Another object of the invention relates to the use of CYFRA in combination with at least LRG-1, ACT, HPT and C9 for the diagnosis and/or management of LC.

A further object of the invention relates to a method for the detection, diagnosis, staging or management of LC in a patient, the method comprising the combined analysis of (i) a CYFRA protein, and (ii) at least a second protein selected from LRG-1, ACT, HPT and C9, in a sample from the subject, said combined analysis providing an indication of the presence, stage or progression of lung cancer in the patient. The term “combined analysis” indicates that the cited markers are tested (e.g., for their presence, amount, relative expression level, variations, etc.) in the sample, either simultaneously or separately. It is the combined measure or variation of the at least two markers which provides a relevant and specific indication of the disease.

In a particular embodiment, the method comprises providing a sample from the subject, contacting said sample, or a derivative thereof, with a first specific binding reagent that binds a CYFRA protein and with at least a second specific reagent that binds a second protein selected from LRG-1, ACT, HPT, and C9, and determining the presence or amount of protein bound to said binding reagents. Contacting with the at least 2 binding reagents can be performed simultaneously or sequentially, on (a same aliquot of) the same sample or on different aliquots of a same sample. The binding reagents are, most preferably, antibodies, fragments or derivatives thereof.

A further object of the invention resides in a method for detecting lung cancer in a subject, the method comprising contacting a sample from said subject, preferably a blood sample, with a first antibody, fragment or derivative thereof, that binds a CYFRA protein and with at least a second antibody, fragment or derivative thereof, that binds a protein selected from LRG-1, ACT, HPT, and C9, and determining the presence of a binding, said presence being indicative of lung cancer.

The invention also relates to a device comprising at least one binding reagent (e.g., an antibody, fragment or derivative thereof) that binds a CYFRA protein and at least one binding reagent (e.g., an antibody, fragment or derivative thereof) that binds a protein selected from Leucine-Rich alpha-2 glycoprotein, haptoglobin, C9, or Alpha 1 Antichymotrypsin, immobilized on a support. Preferably the support is a membrane, a slide, a microarray, a chip or a microbead.

### LEGEND TO THE FIGURES

FIG. 1. A dot-plot showing CYFRA 21-1, ACT, LRG-1, C9 and Hpt as measured by ELISA in plasma samples from healthy controls (n=158) and lung cancer patients (n=230). The statistics are shown as box plots to each group where the bottom and top of the box are the 25^{th} and 75^{th} percentiles; the band in the box is the median value.

FIG. 2. Standard curves of sandwich ELISA assays with (a) anti-ACT antibodies; (b) anti-beta-HPT antibodies; (c) anti-CFH antibodies; (d) anti-LRG antibodies; (e) anti-C9 antibodies.

FIG. 3. Receiver operator curves (ROC) analysis of the diagnostic performance of single biomarkers and combinations of biomarkers.

(a) all stages of LC: blue—LRG, AUC=0.85; black—CYFRA, AUC=0.91; red—CYFRA+LRG, AUC=0.93; green—CYFRA+LRG+ACT+C9+Hpt+CFH, AUC=0.94;

(b) only stage I of LC: blue—LRG, AUC=0.88; black—CYFRA, AUC=0.92; red—CYFRA+LRG, AUC=0.95; green—CYFRA+LRG+ACT+C9+Hpt+CFH, AUC=0.96;

(c) stages II, III and IV of LC: blue—LRG, AUC=0.92; black—CYFRA, AUC=0.91; red—CYFRA+LRG, AUC=0.96; green—CYFRA+LRG+ACT+C9+Hpt+CFH, AUC=0.97;

### DETAILED DESCRIPTION OF THE INVENTION

A challenge in the treatment of lung cancer is the lack of early, pre-symptomatic detection as lung cancer symptoms generally present at advanced stages. Here, we describe biomarker combinations which allow specific, reliable and sensitive detection and staging of lung cancer in human subjects.

International application No PCT/EP2010/061354, presently unpublished, discloses the discovery of cancer specific biomarkers. In the present application, specific combinations of some of these biomarkers with CYFRA were tested and shown to exhibit remarkably high levels of selectivity and specificity for diagnosing and managing lung cancer.

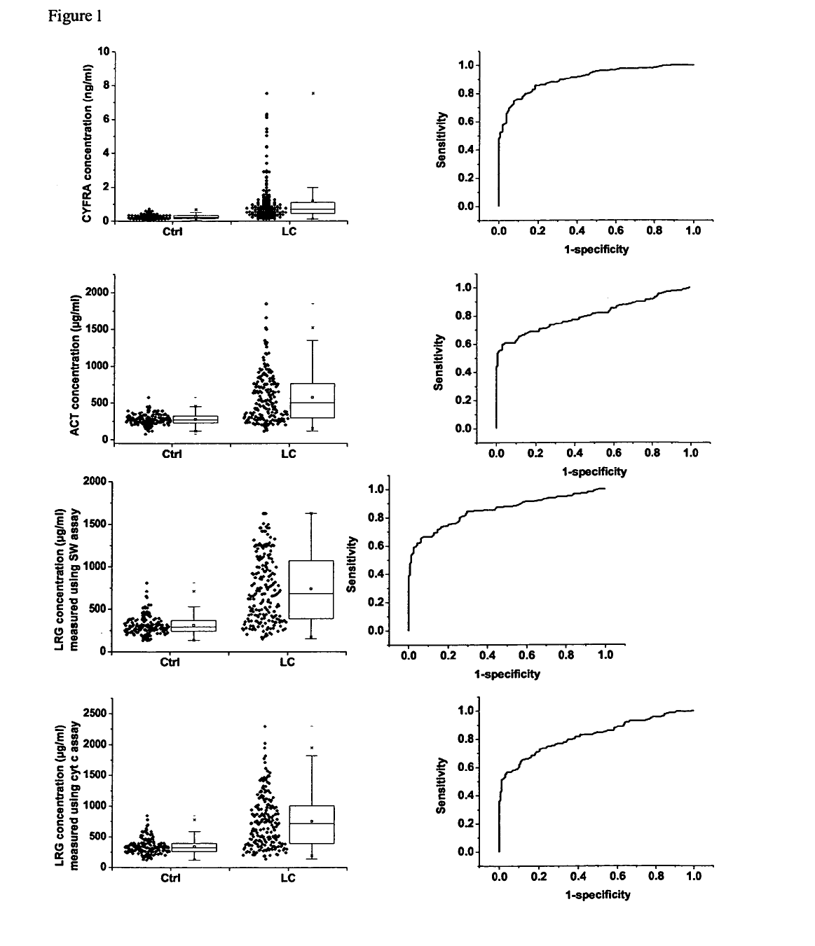

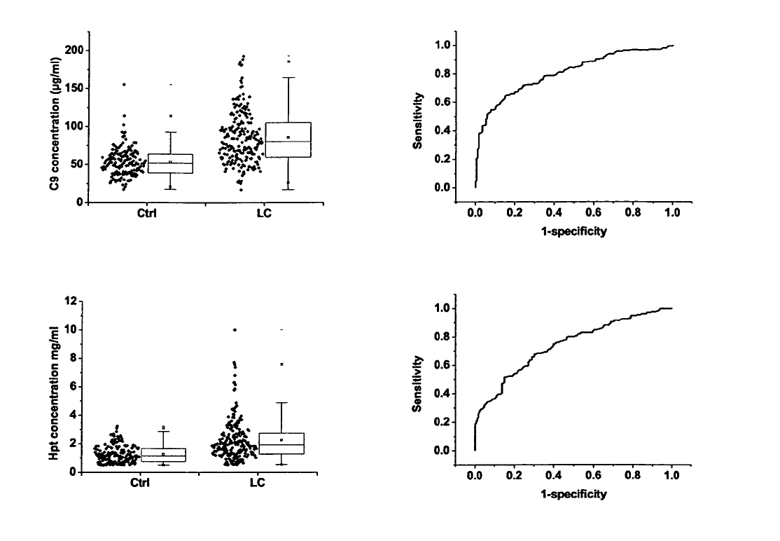

The inventors have measured the concentration of the different biomarkers in plasma of lung cancer patients and healthy subjects using sandwich ELISA assays. HPT, ACT, LRG-1, and C9 proteins were measured using SW ELISA assays (FIG. 1), and a CYFRA protein (e.g., cytokeratin-19 fragment) was measured using a commercially available kit CYFRA 21-1 EIA (Fujirebio). The present invention shows the use of CYFRA, in combination with either one of the above 4 biomarkers or their combinations provides a reliable assessment of the presence, stage or progression of lung cancer in human subjects. Measurement of anyone of these combinations may be used for the early detection or for the follow-up of patients having lung cancer, including NSCLC cancer, with high sensitivity and selectivity. Furthermore, the above biomarker combinations may be used in further combination with additional markers, such as e.g., Complement factor H(CFH), depending on the disease stage. The method is particularly suited to detect stage I and II, more particularly stage Ia and IIa, lung cancer.

Combinations of each of these individual biomarkers with CYFRA, as well as multimember panels of all biomarkers, were tested and showed remarkable diagnostic performance (measured as area under the curve of the ROC), higher than 0.9, and sensitivity up to 84% at 95% specificity.

Altogether, the invention thus provides novel combinatorial biomarkers for clinical applications in lung cancer patient detection, management, diagnosis and monitoring.

The invention thus relates to a method of detecting, diagnosing, staging or managing lung cancer in a subject, based on a combined analysis of at least two markers, a CYFRA protein and at least one second protein selected from LRG-1, ACT, HPT, and C9.

A specific object of the invention resides in a method for the detection, diagnosis, staging or management of lung cancer in a patient, the method comprising the combined analysis of a CYFRA protein and at least a second protein selected from LRG-1, ACT, HPT, and C9, in a sample from the subject, said combined analysis providing an indication of the presence, stage or progression of lung cancer in the patient.

A further object of the invention resides in a method for detecting lung cancer in a subject, the method comprising contacting a sample from said subject, preferably a blood sample, with a first antibody, fragment or derivative thereof, that binds a CYFRA protein and with at least a second antibody, fragment or derivative thereof, that binds a protein selected from LRG-1, ACT, HPT, and C9, and determining the presence of a binding, said presence being indicative of lung cancer.

A further object of the invention resides in a method for detecting lung cancer in a subject, the method comprising determining, in a sample from said subject, preferably a blood sample, the presence or amount of a CYFRA protein and of at least a second protein selected from LRG-1, ACT, HPT, and C9, said presence (or absence) or amount being indicative of lung cancer.

Another object of the invention relates to the use of a CYFRA protein in combination with at least one protein selected from LRG-1, ACT, HPT, and C9, for the detection, diagnosis, staging or management of LC.

Within the context of the invention, the use of a protein for cancer detection, diagnosis or management includes, without limitation, the use of the protein (in any form, soluble or not, full length or not), or of any coding nucleic acids, as a biomarker. This includes, e.g., the use of any reagent to detect or quantify (i) the protein or any variant or mutant thereof, such as splicing variants or polymorphisms, and/or (ii) any nucleic acid encoding said proteins, such as DNA or RNA, said protein and/or nucleic acid levels being correlated to the disease. The term also includes any measure of the expression level of the cited protein, and a comparison of the measured level to a reference or mean value. The measured amount or level or information provides an indication regarding the specified disease in the subject.

### CYFRA

Within the context of this invention, the term CYFRA designates any Cytokeratin protein or any fragment of a cytokeratin protein. More preferably, the term CYFRA designates any soluble or circulating form of a cytokeratin protein or fragment.

Three major cytokeratins are found in simple epithelia: Cytokeratin 19 (GI: 127799941, illustrative sequence is shown in SEQ ID NO: 87 below), the smallest human keratin; Cytokeratin 8, and Cytokeratin 18. Cytokeratin 19 is a major keratin in carcinomas (Chu P G et al., Histopathology 2002, 40:403-439). During apoptosis, soluble cytokeratin fragments are produced from epithelial cells by caspase-mediated proteolysis (Dohmoto K et al., Int J Cancer 2001, 91:468-473). One of these fragments is a fragment of Cytokeratin 19, designated CYFRA21-1, which is produced by cleavage at ²³⁴SVEVD caspase-cleavable sequence, resulting in a 18.4 kDa fragment that corresponds to amino acid residues 234 to 400 of the sequence of SEQ ID NO: 87. CYFRA21-1 was proposed as a biomarker for lung cancer (see e.g., Stieber P., et al., Clin. Biochem., 26: 301-304, 1993, and Cancer (Phila.) 72: 707-713, 1993; Pujol J-L., et al., Cancer Res., 53: 61-66, 1993; or Ebert W et al., Eur. J. Clin. Chem. Clin. Biochem., 32: 189-199, 1994). Recent evidence further show that tumour cells may also release full-length cytokeratin 19 (Alix-Panabieres C et al., Breast Cancer Research Vol. 11, Issue: 3 Article Number: R39, 2009). However, the CYFRA21-1 fragment or full length cytokeratins, alone, do not represent reliable biomarkers sufficient to provide a specific and sensitive LC diagnostic assay.

As indicated above, the inventors have now surprisingly discovered that the use of CYFRA, in combination with either one of C9, HPT, LRG-1, ACT or CFH, or their combinations, provides a reliable assessment of the presence, stage or progression of lung cancer in human subjects. Measurement of anyone of these combinations may be used in particular for the early detection or for the follow-up of patients having lung cancer, including NSCLC cancer, with remarkable diagnostic performance substantially higher than each marker individually, as illustrated e.g., by a sensitivity as high as 84% at 95% specificity.

Detecting a CYFRA protein, or using CYFRA in a method of the invention, designates the detection of any form of CYFRA (e.g., full length or fragments thereof, preferably soluble forms thereof) or corresponding nucleic acids. Specific examples of a CYFRA protein according to the invention include full length Cytokeratin 19, CYFRA21-1 fragment, and any polypeptide comprising a sequence selected from SEQ ID NO: 93 to 96.

The presence or amount of a CYFRA protein can be detected using a binding reagent, particularly a specific antibody (or fragments or derivatives thereof retaining antigen specificity). In a preferred embodiment, the invention uses an anti-CYFRA antibody which binds a human cytokeratin 19 epitope. More preferably, the invention uses an antibody that binds an epitope contained in the amino acid of human cytokeratin 19 represented below. More specific examples are antibodies which bind a peptide selected from SEQ ID NO: 93-96 and which also binds a human cytokeratin protein, or a fragment or derivative of such an antibody having the same antigen specificity. Specific examples of such antibodies include monoclonal antibodies BM19.21 and KS 19.1.

Cytokeratin 19 fragments (e.g., CYFRA21-1) was measured using a commercially available assay based on monoclonal antibodies The epitope mapping of human keratin 19 of the specificities of antibodies BM 19.21 (Böttger V. et al., Eur J Biochem 1995; 231(2):475-85) and KS 19.1 (Achtstatter, T. (1988) Koexpressionsmuster von Intermediiirfilament-Proteinen, University of Heidelberg) using synthetic peptides is shown below:

| SEQ ID | Sequence | Ab |
|---|---|---|
| 93 | DVRADSERQNQEYQR | BM |
| 94 | SERQNQEYQRLMDIK | 19.21 |
| 95 | QEYQRLMDIKSRLEQ | |
| 96 | MKAALEDTLAETEAR | KS 19.1 |

The mapping of the synthetic peptides recognized by the two antibodies on the sequence of human keratin 19 is shown on the following sequence. BM 19.21 recognizes an epitope localized between residues 351 and 375 and KS 19.1 recognizes an epitope localized between residues 316 and 330.

| Amino Acid sequence of Human Cytokeratin 19 (SEQ ID NO: 87): | |
|---|---|
| 1 | MTSYSYRQSS ATSSFGGLGG GSVREGPGVA FRAPSIHGGS GGRGVSVSSA RFVSSSSSGG |
| 61 | YGGGYGGVLT ASDGLLAGNE KLTMQNINDR LASYLDKVRA LEAANGELEV KIRDWYQKQG |
| 121 | PGPSRDYSHY YTTIQDLRDK ILGATIENSR IVLQIDNARL AADDFRTKFE TEQALRMSVE |
| 181 | ADINGLRRVL DELTLARTDL EMQIEGLKEE LAYLKKNHEE EISTLRGQVG GQVSVEVDSA |
| 241 | PGTDLAKILS DMRSQYEVMA EQNRKDAEAW FTSRTEELNR EVAGHTEQLQ MSRSEVTDLR |
| 301 | RTLQGLEIEL QSQLSMKAAL EDTLAETEAR FGAQLAHIQA LISGIEAQLG DVRADSERQN |
| | ₄_______________ ₁__________ |
| | ₂____ |
| 361 | QEYQRLMDIK SRLEQEIATY RSLLEGQEDH YNNLSASKVL |
| | ____ |
| | __________ |
| | ₃_______________ |

Other antibodies may be found or generated against a CYFRA protein and used in the present invention. It should be noted, however, that the use of antibodies that bind an epitope present in any one of SEQ ID NOs: 93-96 is particularly preferred.

### CYFRA/LRG-1 Combinations

The leucine-rich repeat (LRR) family of proteins, including LRG-1, has been shown to be involved in protein-protein interaction, signal transduction, and cell adhesion and development. LRG1 is expressed during granulocyte differentiation (O'Donnell et al., 2002). Human LRG1 was isolated from human serum by Haupt and Baudner, 1977. By sequence analysis, Takahashi et al. (1985) determined that purified LRG1 protein has 312 amino acids and an experimentally determined molecular mass of 45 kD. The LRG1 polypeptide contains 1 galactosamine and 4 glucosamine oligosaccharides attached and has 2 intrachain disulfide bonds. Leucine comprises 66 of the 312 amino acids, and LRG1 contains at least 8 24-amino acid leucine-rich repeats. Increased LRG1 expression was detected in GCSF-treated human cells derived from a patient with myeloproliferative disorder. In contrast, decreased LRG1 expression was detected after PMA treatment and induction of monocytic differentiation of HL-60 cells

A number of proteomics studies focused on plasma and serum have demonstrated an association between elevated levels of leucine rich alpha-2 glycoprotein (LRG1) and the presence of a number of different cancers in multiple preliminary studies. However, none of the findings have been confirmed by clinical validation. All of these studies are based on proteomics and none involved the use of antibodies.

The invention discloses that LRG1, in combination with CYFRA, represents highly predictive lung cancer-specific biomarker combination. In particular, the results obtained demonstrate that a combined analysis of CYFRA and LRG-1 substantially increases the reliability and sensitivity of lung cancer detection. In particular, the combined assessment of CYFRA and LRG-1 allows the detection of lung cancer with a sensitivity of 83.25% at 95% specificity while, individually, CYFRA and LRG-1 provide a sensitivity of 67.39 and 62.26, respectively.

In this respect, a particular object of the invention relates to the use of CYFRA in combination with LRG-1 for the detection, diagnosis and/or management of LC.

Another object of the invention relates to the use of CYFRA in combination with LRG-1 and in further combination with at least a protein selected from ACT, HPT, and C9, for the detection, diagnosis and/or management of LC. The results show that sensitivity of detection may be further increased by adding a limited number selected biomarkers. In particular, the combined assessment of CYFRA, LRG-1, C9 and ACT allows the detection of lung cancer with a sensitivity of 84.24% at 95% specificity. Such a combined assessment represents a preferred embodiment of the invention.

A further object of the invention relates to a method for the detection, diagnosis, staging or management of LC in a patient, the method comprising the combined analysis of a CYFRA protein and an LRG-1 protein, in a sample from the subject, said combined analysis providing an indication of the presence, stage or progression of lung cancer in the patient. In a further preferred embodiment, the method comprises the analysis of at least one further protein selected from ACT, HPT, C9 and CFH.

A further object of the invention resides in a method for detecting lung cancer in a subject, the method comprising contacting a sample from said subject, preferably a blood sample, with a first antibody, fragment or derivative thereof, that binds a CYFRA protein and with at least a second antibody, fragment or derivative thereof, that binds LRG-1, and determining the presence of a binding, said presence being indicative of lung cancer.

The invention also relates to a device comprising at least one binding reagent (e.g., an antibody, fragment or derivative thereof) that binds a CYFRA protein and at least one binding reagent (e.g., an antibody, fragment or derivative thereof) that binds a LRG-1 protein, immobilized on a support. Preferably the support is a membrane, a slide, a microarray, a chip or a microbead.

Any anti-LRG-1 antibody may be used to carry out the present invention. However, in a preferred embodiment, the invention uses an anti-LRG-1 antibody which binds a peptide selected from SEQ ID NO: 26-35 and 57-63 and which also binds a human LRG1 (Leucine Rich Alpha-2 Glycoprotein 1) protein, or a fragment or derivative of such an antibody having the same antigen specificity. The invention particularly uses an antibody which binds a peptide selected from SEQ ID NO: 26-35 and 57-63 and wherein said binding is at least partially displaced by a human LRG-1 protein, or a fragment or derivative of such an antibody having the same antigen specificity. Specific examples of such antibodies include Bsi0392; Bsi0351 and Bsi0352, as disclosed in the experimental section. A further example includes monoclonal antibody 2F5.A2 produced by hybridoma having ATCC accession number PTA-8131 (U.S. Pat. No. 7,416,850). This patent also discloses a specific assay method for detecting LRG-1, using Cytochrome C and antibody 2F5.A2, which may be specifically used for carrying out the present invention.

### CYFRA/Alpha-1-Antichymotrypsin (ACT) Combinations

The invention discloses that ACT, in combination with CYFRA, represents a highly predictive lung cancer-specific biomarker combination. In particular, the results obtained demonstrate that a combined analysis of CYFRA and ACT substantially increases the reliability and sensitivity of lung cancer detection. More specifically, the inventors have discovered that the combined assessment of CYFRA and ACT allows the detection of lung cancer with a sensitivity of 81.28% at 95% specificity while, individually, CYFRA and ACT provide a sensitivity of 67.39 and 60.85, respectively.

In this respect, a particular object of the invention relates to the use of CYFRA in combination with ACT for the detection, diagnosis and/or management of LC.

Another object of the invention relates to the use of CYFRA in combination with ACT and in further combination with at least a protein selected from LRG-1, HPT, and C9, for the detection, diagnosis and/or management of LC. The results show that sensitivity of detection may be further increased by adding a limited number selected biomarkers. In particular, the combined assessment of CYFRA, LRG-1, C9 and ACT allows the detection of lung cancer with a sensitivity of 84.24% at 95% specificity.

A further object of the invention relates to a method for the detection, diagnosis, staging or management of LC in a patient, the method comprising the combined analysis of a CYFRA protein and an ACT protein, in a sample from the subject, said combined analysis providing an indication of the presence, stage or progression of lung cancer in the patient. In a further preferred embodiment, the method comprises the analysis of at least one further protein selected from LRG-1, HPT, C9 and CFH.

A further object of the invention resides in a method for detecting lung cancer in a subject, the method comprising contacting a sample from said subject, preferably a blood sample, with a first antibody, fragment or derivative thereof, that binds a CYFRA protein and with at least a second antibody, fragment or derivative thereof, that binds ACT, and determining the presence of a binding, said presence being indicative of lung cancer.

The invention also relates to a device comprising at least one binding reagent (e.g., an antibody, fragment or derivative thereof) that binds a CYFRA protein and at least one binding reagent (e.g., an antibody, fragment or derivative thereof) that binds an ACT protein, immobilized on a support. Preferably the support is a membrane, a slide, a microarray, a chip or a microbead.

Any anti-ACT antibody may be used to carry out the present invention. However, in a preferred embodiment, the invention uses an anti-ACT antibody which binds a peptide selected from SEQ ID NOs: 1-13 and 39-42 and which also binds a human Alpha-1-antichymotrypsin protein, or a fragment or derivative of such an antibody having the same antigen specificity. The invention particularly uses an antibody which binds a peptide selected from SEQ ID NOs: 1-13 and 39-42 and wherein said binding is at least partially displaced by a human Alpha-1-antichymotrypsin protein, or a fragment or derivative of such an antibody having the same antigen specificity. A particularly preferred antibody of the invention binds a peptide selected from SEQ ID NOs: 1-5 and 7-12 and also binds a human Alpha-1-antichymotrypsin protein, or a fragment or derivative of such an antibody having the same antigen specificity. Specific examples of such antibodies include Bsi0358, Bsi0359, and Bsi0186, which are disclosed in the experimental section.

### CYFRA/Complement Factor 9 (C9) Combinations

Complement C9 is a component of the complement system, a multi-protein biochemical cascade which aids to clear pathogens. The cascade is activated upon binding of IgG or IgM to pathogen molecules. C9 is one of the terminal components of the cascade and is responsible for forming pores in target cells leading to their destruction. Deficiencies in complement proteins are believed to be linked to auto-immunity and higher sensitivity to infections. The cDNA coding for C9 was sequenced and the protein sequence—537 amino acids in a single polypeptide chain—was derived. The amino-terminal half of C9 is predominantly hydrophilic and the carboxyl-terminal half is more hydrophobic. The amphipathic organization of the primary structure is consistent with the known potential of polymerized C9 to penetrate lipid bilayers and cause the formation of transmembrane channels as part of the lytic action of MAC. Marazziti et al. (1988) compared gene and protein structure of C9 and compared both with low density lipoprotein receptor (606945). The C9 gene is composed of 11 exons with lengths between 100 and 250 bp, except for exon 11 which extends over more than 1 kb, as it includes the 3-prime untranslated region. Witzel-Schlomp et al. (1997) gave revised information on the structure of the C9 gene, especially the exon-intron boundaries

C9 has not been associated with cancer of any type.

The invention discloses that C9, in combination with CYFRA, represents highly predictive lung cancer-specific biomarker combination. In particular, the results obtained demonstrate that a combined analysis of CYFRA and C9 substantially increases the reliability and sensitivity of lung cancer detection. More specifically, the inventors have discovered that the combined assessment of CYFRA and C9 allows the detection of lung cancer with a sensitivity of 76.85% at 95% specificity while, individually, CYFRA and C9 provide a sensitivity of 67.39 and 43.87, respectively.

In this respect, a particular object of the invention relates to the use of CYFRA in combination with C9 for the detection, diagnosis and/or management of LC.

Another object of the invention relates to the use of CYFRA in combination with C9 and in further combination with at least a protein selected from LRG-1, ACT, and HPT, for the detection, diagnosis and/or management of LC. The results show that sensitivity of detection may be further increased by adding a limited number selected biomarkers. In particular, the combined assessment of CYFRA, LRG-1, C9 and ACT allows the detection of lung cancer with a sensitivity of 84.24% at 95% specificity.

A further object of the invention relates to a method for the detection, diagnosis, staging or management of LC in a patient, the method comprising the combined analysis of a CYFRA protein and an C9 protein, in a sample from the subject, said combined analysis providing an indication of the presence, stage or progression of lung cancer in the patient. In a further preferred embodiment, the method comprises the analysis of at least one further protein selected from LRG-1, ACT, HPT, and CFH.

A further object of the invention resides in a method for detecting lung cancer in a subject, the method comprising contacting a sample from said subject, preferably a blood sample, with a first antibody, fragment or derivative thereof, that binds a CYFRA protein and with at least a second antibody, fragment or derivative thereof, that binds a C9 protein, and determining the presence of a binding, said presence being indicative of lung cancer.

The invention also relates to a device comprising at least one binding reagent (e.g., an antibody, fragment or derivative thereof) that binds a CYFRA protein and at least one binding reagent (e.g., an antibody, fragment or derivative thereof) that binds a C9 protein, immobilized on a support. Preferably the support is a membrane, a slide, a microarray, a chip or a microbead.

Any anti-C9 antibody may be used to carry out the present invention. However, in a preferred embodiment, the invention uses an antibody which binds a peptide selected from SEQ ID NOs: 14-25 and which also binds a human C9 (Complement component 9) protein, or a fragment or derivative of such an antibody having the same antigen specificity. The invention particularly uses an antibody which binds a peptide selected from SEQ ID NOs: 14-25 and wherein said binding is at least partially displaced by a human C9 protein, or a fragment or derivative of such an antibody having the same antigen specificity. Specific examples of such antibodies include Bsi0272 and Bsi0452, as disclosed in the experimental section.

### CYFRA/Haptoglobin (HPT)- or Haptoglobin Related Protein (HRPT) Combinations

Haptoglobin (HPT), NM 005143 is a tetrameric protein that functions to bind free plasma hemoglobin, thereby allowing degradative enzymes to gain access to the hemoglobin, while at the same time preventing loss of iron through the kidneys and protecting the kidneys from damage by hemoglobin. Mutations in the HP gene and/or its regulatory regions cause ahaptoglobinemia or hypohaptoglobinemia.

An increase in haptoglobin levels or changes in HPT glycosylation have been associated with almost major forms of cancer.

The invention discloses that HPT, in combination with CYFRA, represents predictive lung cancer-specific biomarker combination. In particular, the results obtained demonstrate that a combined analysis of CYFRA and HPT substantially increases the reliability and sensitivity of lung cancer detection. More specifically, the inventors have discovered that the combined assessment of CYFRA and HPT allows the detection of lung cancer with a sensitivity of 69.46% at 95% specificity while, individually, HPT provides a sensitivity of 32.08.

In this respect, a particular object of the invention relates to the use of CYFRA in combination with HPT for the detection, diagnosis and/or management of LC.

Another object of the invention relates to the use of CYFRA in combination with HPT and in further combination with at least a protein selected from LRG1, ACT, C9 and CFH, for the detection, diagnosis and/or management of LC.

A further object of the invention relates to a method for the detection, diagnosis, staging or management of LC in a patient, the method comprising the combined analysis of a CYFRA protein and an HPT protein, in a sample from the subject, said combined analysis providing an indication of the presence, stage or progression of lung cancer in the patient. In a further preferred embodiment, the method comprises the analysis of at least one further protein selected from LRG-1, ACT, C9 and CFH.

A further object of the invention resides in a method for detecting lung cancer in a subject, the method comprising contacting a sample from said subject, preferably a blood sample, with a first antibody, fragment or derivative thereof, that binds a CYFRA protein and with at least a second antibody, fragment or derivative thereof, that binds an HPT protein, and determining the presence of a binding, said presence being indicative of lung cancer.

The invention also relates to a device comprising at least one binding reagent (e.g., an antibody, fragment or derivative thereof) that binds a CYFRA protein and at least one binding reagent (e.g., an antibody, fragment or derivative thereof) that binds an HPT protein, immobilized on a support. Preferably the support is a membrane, a slide, a microarray, a chip or a microbead.

Any anti-HPT antibody may be used to carry out the present invention. However, in a preferred embodiment, the invention uses antibodies which bind a peptide selected from SEQ ID NOs: 36-38 and which also bind a human haptoglobin or haptoglobin-related protein, or a fragment or derivative of such an antibody having the same antigen specificity. The invention particularly uses an antibody which binds a peptide selected from SEQ ID NOs: 36-38 and wherein said binding is at least partially displaced by a human haptoglobin or haptoglobin-related protein, or a fragment or derivative of such an antibody having the same antigen specificity. Specific examples of such antibodies include Bsi0033, Bsi1709, and Bsi0071, as disclosed in the experimental section.

### CYFRA, LRG-1, C9 and ACT Combination

In a preferred embodiment, the invention comprises the combined determination of CYFRA, LRG-1, C9 and ACT. Indeed, the results show that the sensitivity of detection may be optimal using such a combination CYFRA, LRG-1, C9 and ACT, especially for detection of stage I LC. A specific object of the invention thus resides in a method of detection, diagnosing, staging or monitoring LC in a human subject, the method comprising the combined determination of CYFRA, LRG-1, C9 and ACT in a sample from the subject, said determination being indicative of LC. Another objet of the invention also resides in a device comprising reagents for combined detection of CYFRA, LRG-1, C9 and ACT. A further object of the invention relates to a kit comprising reagents for combined detection of CYFRA, LRG-1, C9 and ACT. Furthermore, although very predictive in itself, this marker combination of the invention may be further combined with other biomarker(s).

### Assay Format and Implementation

The invention may be performed using different types of samples, various types of antibodies or binding reagents, and different assay formats.

The sample may be any biological fluid, including without limitation serum, plasma, blood, urine, cerebrospinal fluid, bronchoalveolar lavage (BAL) fluid, sputum, tear, sweat, amniotic fluid and inflammatory exudates. A preferred embodiment uses blood, serum or plasma, particularly whole blood. The sample may be treated prior to being used in the method of the invention, e.g., for dilution, enrichment, concentration, filtration, etc. Also, various aliquots of a sample may be prepared, for separate testing.

The invention preferably utilises binding reagents that specifically or selectively bind to a biomarker. Selective or specific binding indicates that binding to another molecule can be discriminated from (e.g., occurs with higher affinity or avidity than) specific binding to the target biomarker. Preferred reagents do not bind, under selective condition, to any other unrelated human blood protein but the reference protein. Binding of a reagent to a reference molecule can be tested as disclosed in the examples.

The binding reagents may be selected from aptamers, specific ligands, antibodies, or derivatives thereof.

In a particular embodiment, the binding reagent is an antibody. The antibody may be a polyclonal or a monoclonal antibody, most preferably a monoclonal. It may be of various classes (e.g., IgG, IgE, IgM, etc.). The antibody may be of various animal origin, or human or synthetic or recombinant. Furthermore, the term antibody also includes fragments and derivatives thereof, in particular fragments and derivatives of said monoclonal or polyclonal antibodies having substantially the same antigenic specificity. Antibody fragments include e.g., Fab, Fab′2, CDRs, etc. Derivatives include humanized antibodies, human antibodies, chimeric antibodies, poly-functional antibodies, Single Chain antibodies (ScFv), etc. These may be produced according to conventional methods, including immunization of an animal and collection of serum (polyclonal) or spleen cells (to produce hybridomas by fusion with appropriate cell lines).

Methods of producing polyclonal antibodies from various species, including mice, rodents, primates, horses, pigs, rabbits, poultry, etc. may be found, for instance, in Vaitukaitis et al. [29]. Briefly, the antigen is combined with an adjuvant (e.g., Freud's adjuvant) and administered to an animal, typically by sub-cutaneous injection. Repeated injections may be performed. Blood samples are collected and immunoglobulins or serum are separated.

Methods of producing monoclonal antibodies from various species as listed above may be found, for instance, in Harlow et al (Antibodies: A laboratory Manual, CSH Press, 1988) or in Kohler et al (Nature 256 (1975) 495), incorporated therein by reference. Briefly, these methods comprise immunizing an animal with the antigen, followed by a recovery of spleen cells which are then fused with immortalized cells, such as myeloma cells. The resulting hybridomas produce the monoclonal antibodies and can be selected by limit dilutions to isolate individual clones. Antibodies may also be produced by selection of combinatorial libraries of immunoglobulins, as disclosed for instance in Ward et al (Nature 341 (1989) 544).

Recombinant antibodies, or fragments or derivatives thereof, may be produced by methods known per se in the art, for example by recombination in a host cell, transformed with one or more vectors enabling the expression and/or secretion of the nucleotide sequences encoding the heavy chain or the light chain of the antibody. The vector generally contains a promoter, translation initiation and termination signals, and suitable transcriptional regulatory regions. It is stably maintained in the host cell and may optionally possess specific signals for secretion of the translated protein. These different components are selected and optimized by one of skill in the art according to the host cell used.

For use in the invention, the antibodies may be coupled to heterologous moieties, such as labels, tags, linkers, etc.

The method of the invention may be carried out using a variety of detection technologies or platforms known per se in the art such as, without limitation Capture assay, Sandwich assay, Competition assay, Radio-immuno assays, Enzyme labels with substrates that generate colored, fluorescent, chemiluminescent, or electrochemically-active products, Fluorescence, fluorescent polarization, Chemiluminescence, Optical and colorimetric, Electrochemiluminescence, Time-resolved fluorescence, Surface plasmon resonance, Evanescent wave, Multiwell plate (ELISA), Individual assay, Multiplex assay, Latex bead—multiplex assay, Microarray (Laminar surface)—multiplex assay, Glass, Ceramic (like Randox), Plate based assays, Strip based assays, dipsticks, Closed systems immunoassays.

### Preferred Assay Formats Include:

### Capture Assay

An assay carried out using a single immobilized antibody (multiwall plate, latex bead, microarray, etc.) which captures a specific labeled protein from a biofluid, the detection which is measured using appropriate detection reagents as detailed in the following paragraph.

The antibody is immobilized directly to the support or captured by an affinity reagent such as an anti-mouse IgG antibody coated onto the support. The immobilized antibody is then incubated with any of the above mentioned body fluids in which the proteins have been labeled with a detection molecule such as biotin, with or without pre-treatment to remove abundant proteins. The labeled protein which is bound by the antibody is detected by the addition of an appropriate detection reagent which binds to the label such as avidin or streptavidin which has been modified to be compatible with one of the detection technologies described in the section “detection technology.”

The resulting signal provides a quantitative measure of the amount of labeled protein bound by the antibody

### Sandwich ELISA

An assay using two antibodies, the first which is immobilized on a support (multiwell plate, latex bead, microarray, etc.) which binds a specific protein from a biofluid, the detection which is measured using a labeled second antibody against the same protein and appropriate detection reagents as detailed in the following paragraph.

The first antibody is immobilized directly to the support or captured by an affinity reagent such as an anti-mouse IgG antibody coated onto the support. The immobilized antibody is then incubated with any of the above mentioned body fluids, with or without pre-treatment to remove abundant proteins. The antibody/antigen complex is then incubated with a second antibody, made against the same protein, which has been labeled with a detection molecule such as biotin. The bound antibody is detected by the addition of an appropriate detection reagent which binds to the label such as avidin or streptavidin which has been modified to be compatible with one of the detection technologies described in the section “detection technology.”

The resulting signal provides a quantitative measure of the amount of protein bound by the antibody

### Competitive Assay

An assay in which the binding of a labeled tracer protein by a single antibody as described in “capture assay” is inhibited by pre-incubation of a biofluid to indirectly quantify the analyte.

The antibody is immobilized directly to the support or captured by an affinity reagent such as an anti-mouse IgG antibody coated onto the support. The immobilized antibody is then incubated with any of the above mentioned body fluids. The immobilized antibody/antigen complex is then incubated with a labeled tracer consisting of either (1) any of the above mentioned body fluids in which the proteins have been labeled with a detection molecule such as biotin, with or without pre-treatment to remove abundant proteins, or (2) a purified or recombinant protein recognized (bound) by the monoclonal antibody, or (3) a peptide which is recognized (bound) by the monoclonal antibody. The labeled protein or peptide which is bound by the antibody is detected by the addition of an appropriate detection reagent which binds to the label such as avidin or streptavidin which has been modified to be compatible with one of the detection technologies described in the section “detection technology.”

The level of the specific protein in the unlabeled biofluid is determined as a function of the inhibition of signal.

Preferred Detection Technologies include:

Enzyme labels with substrates that generate colored, fluorescent, chemiluminescent, or electrochemically-active products.

The detection reagent (for example steptavidin or avidin, which binds to biotin) is coupled to an enzyme such as horseradish peroxidase which is capable of catalyzing
an appropriate colorimetric substrate of which the product demonstrates maximal absorbance at a given wavelength allowing the quantitative measurement of the labeled protein by measuring the optical density of the final product in the well at or near the wavelength of maximal absorbance.
a chemiluminescent substrate to a sensitized reagent which upon oxidation emits light, providing the quantitative measurement of the labeled protein.
a chemiluminescent substrate to a sensitized reagent which upon the application of an electrical current emits light, providing the quantitative measurement of the labeled protein

### Fluorescence

The detection reagent (for example steptavidin or avidin, which binds to biotin) is coupled to a fluorescent tag.

Preferred platform Technologies include:

### Multiwell Plate

Single test: one antibody is immobilized per well either directly or indirectly using a capture reagent such as goat anti-mouse antibody.
Multiplex: 2 or more antibodies are immobilized in a single well by deposition in a pattern

### Latex Bead

Two or more antibodies are immobilized onto a latex bead between x and y microns

### Arrays, Microarrays, and Nanoarrays

Two or more antibodies are spotted onto an activated laminar surface with a spot diameter between 100 μm-5 mm (arrays), 2 μm-100 μm (microarrays), 10 nm-2 μm (nano-arrays) The surface can be composed of glass, plastic, ceramic, carbon nanotube lattice etc.

Different methods or algorithms can be used to derive the lung cancer index. In the simplest form, the concentrations of the individual markers measured in the sample are combined using a linear function (see below) to derive an index value which indicates the presence of lung cancer if the index value is above a pre-determined threshold.

Index=A*Concentration(BM1)+B*Concentration(BM2)+C*Concentration(BM3) . . .

Where BM designates a biomarker.

Another method or algorithm consists of first proceeding to the standardization of the obtained individual concentration measurements using pre-determined values for the mean and the standard deviation (std) (see below) for each biomarker as to bring the measurements of biomarkers in different concentration ranges to the same scale, as follows:

Standardized Value(BM1)=((Concentration(BM1)−Mean(BM1))/Std(BM1))

The standardized values are then used as described in the previous paragraph. The standardization procedure can also be used to correct for reproducible changes in the level of the biomarker concentrations that can be attributed to analytical factors.

The method of the invention can be performed at any stage of the disease, such as early or late stage, to confirm or reject a prior diagnosis, select patients for surgery, classify cancer type or severity, or monitor patients. The test may also be conducted before disease symptoms, as a first line detection. Clinical diagnostic application of a lung cancer plasma (serum) test will be useful e.g., for patients who are suspected to have lung cancer because of a suspicious nodule that has been detected by imaging of the lung (CT scan). Nodules<0.5 cm are suspicious and quite frequent in the populations. Nodules>0.5 cm, but <1.1 cm represent an increased likelihood of being cancerous, however challenging to find by surgery and invasive endoscopic procedures. It is important to select patients from this group with nodules that are definitively cancerous to reduce the burden of futile surgeries and other invasive diagnostic procedures.

The test will avoid futile thoracotomies and unnecessary and expensive imaging technologies that are not specific enough and expose the patients to potentially harmful irradiation, and missed cures as observed patients could receive the test repeatedly.

The invention is particularly advantageous as it allows detection of Stage I or II, in particular Stages Ia and stage IIa LC in human subjects.

Further aspects and advantages of the invention will be disclosed in the following examples, which should be considered illustrative.

### EXAMPLES

### A. Methods

### Clinical Samples

Plasma samples from patients with newly diagnosed lung cancer and no previous treatment were obtained from Asterand (Royston, UK) and from the Department of Pulmonology of the University of Debrecen in Hungary from informed patients and apparently healthy individuals after obtaining their written consent by a clinical protocol RKEB/IKEB:2422-2005 approved by the regional ethics committee and the IRB of the clinic (Table 1). Lung cancer staging was done according to the American Joint Committee on Cancer (AJCC) and was based on information in the final histopathology report having the LC-histotype according to the WHO classification (19). Clinical data including stage at diagnosis, histology, additional pulmonary pathologies, smoking habits and general patient demographics are presented in Table1.

| | | Number | | | | packs/day | | | |
|---|---|---|---|---|---|---|---|---|---|
| Cohort | Status | subjects | Age | Gender* | Smoking status | (avg) | Stage LC | Histology^{†} | Other LD^{‡} |
| Training set | LC | 219 | 26-86 | M: 158 | Current Use: 116 | 1.3 | I: 128 | SQC: 104 | COPD: 6 |
| (Asterand) | | | (avg | F: 61 | Occasional use: 5 | | II: 39 | AC: 85 | Emphysema: 5 |
| | | | 61) | | Previous use: 63 | | III: 31 | Other | Chronic |
| | | | | | Never used: 32 | | IV: 5 | NSCLC: 25 | bronchitis: 21 |
| | | | | | unknown: 3 | | unknown: | SCLC: 5 | Asthma: 3 |
| | | | | | | | 16 | | |
| | Healthy | 169 | 18-70 | M: 114 | Current Use: 49 | 1.0 | — | — | — |
| | | | (avg | F: 55 | Occasional use: 4 | | | | |
| | | | 38) | | Previous use: 25 | | | | |
| | | | | | Never used: 91 | | | | |
| Test set Univ. | LC | 45 | 44-77 | M: 32 | Current Use: 45 | 1.7 | I: 1 | SQC: 14 | COPD: 22 |
| Of Debrecen | | | (avg | F: 13 | | | III: 1 | AC: 10 | |
| | | | 59) | | | | III-IV: 12 | Other | |
| | | | | | | | IV: 18 | NSCLC: 4 | |
| | | | | | | | unknown: | SCLC: 17 | |
| | | | | | | | 13 | | |
| | Healthy | 63 | 34-72 | M: 23 | Current Use: 63 | 1.1 | — | — | COPD: 0 |
| | | | (avg | F: 40 | | | | | |
| | | | 54) | | | | | | |

### Antibody Purification

Ascites fluid was produced by intraperitoneally injecting 1-5×10⁶ cloned hybridoma cells into incomplete Freund's adjuvant-primed Balb/c mice, using a protocol which was approved by the animal care committee of the animal facility. Ascitic fluid was cleared of cells and cell debris, and IgG was purified using a two-step affinity chromatography procedure consisting of pre-purification on a Pierce thiophilic adsorbent column from Thermo Fisher Scientific (Waltham, Mass.) followed by further purification using Protein-G Sepharose 4 FF from GE Healthcare (Chalfont St. Giles, UK). In both cases the procedures were carried out as recommended by the suppliers. The purified antibodies were concentrated by ultrafiltration. The purities of the preparations were assessed by SDS-PAGE, and the protein concentration was determined using the BCA protein assay kit from Pierce (Rockford, Ill.). The purified antibodies were stored as working aliquots at −78° C.

Biotinylation of mAbs

Purified monoclonal antibodies were labeled with a bifunctional NHS-biotin having a long alkyl chain as a spacer EZ-Link Sulfo-NHS-LC-Biotin from Pierce (Rockford, Ill.). Labeling was performed in PBS buffer (pH 7.0) at a 50 time molar excess of biotin for 30 min. at room temperature.

### Biomarker Quantification

The different biomarkers were quantified with sandwich ELISA assays using the Abs couples listed in the Table below. The 384-well microtiter plates (high binding plates from Corning) were directly coated with the capture mAbs at 20 μg/ml and then blocked with PBS buffer containing 0.5% BSA (w/v). The plasmas and the protein calibrators, diluted in PBS containing 0.05% (v/v) Tween 20 and 1% FBS (v/v), were then incubated in the wells. The binding of the biotinylated detection antibodies, added at 5 μg/ml and washed 4 times with PBS containing 0.05% (v/v) Tween 20 was then followed by using an avidin complex coupled to the HRP (VECTASTAIN® ABC kits) and its substrate (TMB). The reaction was measured using kinetic readings at 650 nm during 2 min at 37° C.

LRG has been also quantified with a second type of assay based on the affinity of the LRG for the Cytochrome C using the protocol described in Weivoda et al. (J Immunol Methods. 2008 Jul. 20; 336(1):22-9. Epub 2008 Apr. 4).

The standard curves obtained with the calibrators have been plotted using the four-parameter fitting algorithm. The concentrations of the analytes in the plasmas were calculated from the corresponding standard curves.

CYFRA21-1 was measured using a commercially available assay based on monoclonal antibodies BM 19.21 and KS 19.1 (Cyfra 21-1 EIA kit from Fujirebio Diagnostics, Inc. (ref. 211-10).

The monoclonal antibodies and reagents used in this work are listed below:

| Capture reagent | Detection Ab | Target Protein |
|---|---|---|
| BM 19.21 | KS 19.1 | CYFRA21-1 |
| Bsi0272 | Bsi0452 | C9 |
| Bsi0352 | 2F5.A2* | LRG-1 |
| Cytochrome c | Bsi0392 | LRG-1 |
| Bsi1709 | Bsi0033 | Haptoglobin |
| Bsi0186 | Bsi0358 | ACT |

*Weivoda S, et al., J Immunol Methods. 2008 Jul. 20; 336(1): 22-9

Immunisation of BALB/c Mice with Complex Immunogens

Female Balb/c mice of at least 8 weeks of age from Charles River Laboratories (Evry, France) were injected subcutaneously in the rear footpads and at the base of the tail with the two complex antigen protein mixtures. Each mice received 10 μg protein of complex immunogen preparation on days 1, 15 and 29. Complete Freund's adjuvant (Sigma, Saint Louis, Mo.) was used in all cases. Blood was taken from each mouse by retro-orbital bleed using a Pasteur pipette on days 19 and 33 to monitor antibody production by ELISA. Three weeks minimum after the third immunization (day 52 for group A and day 61 for group B) an additional injection with 10 μg of the complex antigen mixture in PBS pH 7.0 was performed to boost the immune response. Mice were treated in an animal facility according to the French and European laws and regulations regarding animal experimentation.

### Fusion, Cell Growth and Cloning

The procedures were done according to published methods. In short; Sp2/0-Ag-14 cells were fused to splenocytes of immunized mice with the help of polyethylene glycol (PEG). Fused cells were seeded to provide quasi clonal distribution of hybrids, which were selected in HAT media. Hybridoma cell lines were cloned before antibody production (in-vitro or in-vivo)

### High Throughput Direct-ELISA Screening

All chemicals, unless specified, were obtained from Sigma (St. Louis, Mo.). 384 well high-binding plates (Corning Inc., Lowell, Mass.) were coated with 20 μg/ml (13 μl per well) goat anti-mouse Ig gamma chain specific polyclonal antibody (GAM) from Southern Biotechnology Associates, Inc. (Birmingham, Ala.) in coating buffer at pH 9.6 for 2 h at room temperature (RT). The plates were washed four times with 80 μl/well of PBS containing 0.05% (v/v) Tween (washing buffer) and blocked with 40 μl of PBS and 0.5% bovine serum albumine (BSA) at 4° C. overnight. Supernatants from the nascent hybridomas or from the clonal cell lines were added non-diluted to the wells. Each hybridoma supernatant was added to four adjacent wells to provide four independent readings. Mouse anti-human mAb against albumin was used as positive control and spotted in eight wells at 1.2 μg/ml in CM (13 μl/well). CM was used as negative control and also added to eight wells. The plates were incubated and then washed four times with washing buffer. All wells were incubated with biotinylated depleted plasmas (tracers) at 10 μg/ml in PBS with 0.05% (v/v) Tween and 1% low IgG FBS. Incubation was continued for 90 min. at RT and the unbound proteins were removed by washing the plates four times with washing buffer. HRP-coupled avidin (Vectastain Elite ABC Peroxidase kit from Reactolab SA, Switzerland) was used as specified by the vendor's protocol. After four fold washing with washing buffer, reaction development was carried out by adding 20 μl freshly prepared substrate solution to each well (o-phenylenediamine at 0.4 mg/ml in 0.05 M phosphate/citrate buffer pH 5.0). The kinetics of the reaction development at 37° C. was followed at 450 nm by recording the absorbance multiple times. Liquid handling was performed using Multidrop Combi from Thermo (Waltham, Mass.), Multimek with 96 pin head from Beckman Coulter (Fullerton, Calif.) and STAR from Hamilton (Reno, Nev.). Plate washing was performed using ELX405 from BioTek (Winooski, Vt.). Absorbance was measured with a microplate reader SpectraMax from Molecular Device (MDS, Toronto, Canada).

### Screening Data Analysis.

Vmax of the chromogenic reactions were calculated from the linear part of the kinetic readings using the software provided with the plate reader SoftMax Pro from Molecular Device (MDS, Toronto, Canada). Each plate had eight positive and negative controls used to calculate Z′ factor, a metrics used to quantify the quality of the screening experiment with respect to reproducibility and data scatter¹⁹. Plates with a Z′ factor below 0.5 (usually less than 10% in a screening campaign) were repeated. The positive (PC) and negative controls (NC) were used to normalize the data across plates and according to the following formula:

VmaxNₛₐₘₚₗₑ=(Vmaxₛₐₘₚₗₑ−Vmax_{NC})/(Vmax_{PC}−Vmax_{NC}).

Aberrant data (outliers) for each group of replicates (four per hybridoma sample reacted with one tracer and eight per control reacted with one tracer) were removed using automated procedure based on the mean and standard deviation values of the multiple measurements.

For each sample (i), the coefficient of variation (CV) is calculated on the n replicates. CV is a normalized measure of dispersion of the probability distribution and it is defined as the ratio of the standard deviation σ to the mean μ as follows:

CV(i)=σ(i)/μ(i)

If CV>0.05, then a maximum (Tₘₐₓ) and a minimum (Tₘᵢₙ) thresholds are calculated as follows:

Tₘₐₓ(i)=μ(i)+1.3*σ(i)

Tₘᵢₙ(i)=μ(i)−1.3*σ(i)

The replicates which VmaxN is lower than Tₘᵢₙ or higher than Tₘₐₓ are removed. In total 9.2% of all generated data was considered as aberrant which represents the removal of less than one measurement for every two samples. The data obtained after normalizing and averaging the replicates (FIG. 6) were further analyzed using statistical methods.

### Statistical Methods

The normality of the distribution of the results was estimated using the Wilks-Shapiro test. The distribution of the results for each hybridoma was calculated separately for the control and the lung cancer samples. Nonparametric statistical analyses were applied: differences between two independent groups were determined using the Mann-Whitney U test; differences between more than two groups were assessed using the Kruskal-Wallis one-way analysis of variance test. All statistical tests were two-sided and were performed using R statistical software (www.cran.r-project.org). A predictive model for discriminating lung cancer cases from healthy controls using the panel of five mAbs, was produced using the freely-available machine learning toolkit Weka (http://www.cs.waikato.ac.nz/˜ml/) with a linear support vector machine and sequential minimal optimization algorithm (SMO). The model was established on the entire dataset using 10 fold cross validation.

Logistic regression based on the SMO predictions was calculated in order to produce probabilities of class values (here lung cancer vs. control) and to generate the Receiver Operating Characteristics (ROC) curves.

### B—Anti-LRG-1 Antibodies

### Bsi0392

Bsi0392 is an IgG type monoclonal antibody. The heavy chain variable region amino acid sequence is represented in SEQ ID NO: 92, which is reproduced below (CDRs are underlined):

QIQLVQSGPELKKPGETVEISCKASGYTFTDYSMHWVKQAPGKGLKWMGW
INTETGEPTYADDFKGRFAFSLETSATTAYLQINNLKNEDTATYFCARGG
YYGNYDYAMDYWGQGTSLTVSS

### Bsi0352

Bsi0352 is an IgG type monoclonal antibody. The heavy chain variable amino acid sequence is represented in SEQ ID NO: 89, which is reproduced below (CDRs are underlined):

EVQLQESGPSLVKPSQTLSLTCSVTGDSITSGSWNWIREFPGNKLEYMGY
ISYSGSTDYSPSLKSRISITRDTSKNQYYLQLNSVTTEDTATYYCATHYY
GYLSLDYWGQGTSVTVSS

The difference in biomarker level with Bsi0352 is represented FIG. 1, showing a very substantial difference between control and lung cancer. Peptides bound by Bsi0352 have been identified and verified. These peptides are presented as SEQ ID NOs: 26-35.

### Bsi0351

Bsi0351 is an IgG type monoclonal antibody. The heavy chain variable amino acid sequence is represented in SEQ ID NO: 88, reproduced below (CDRs underlined).

DVQLQESGPGLVKPSQSLSLTCTVTGYSIINDYAWNWIRQFPGNKLEWMA
YISYGGSIGYKPSLKSRISITRDTSKNQFFLQLNSVTTEDTATYYCARGG
FYALDYWGQGTS

Peptides bound by Bsi0351 have been identified. These peptides are presented as SEQ ID NOs: 57-63.

### C—Anti-ACT Antibodies

Bsi0358

Bsi0358 is an IgG type monoclonal antibody. The heavy chain variable amino acid sequence is represented in SEQ ID NO: 90 reproduced below (CDRs underlined).

EVQLVESGGGLVQPKGSLKLSCAASGFTFNTYAMSWVRQAPGKGLEWISR
IRSKSNNYATYYVDSVKDRFTIYRDDSQSMLYLQMNNLKIEDTAIYYCVR
EGDWGQGTLVTVSA

The difference in biomarker level with Bsi0358 is represented FIG. 1, showing a very substantial difference between control and lung cancer. Peptides bound by Bsi0358 have been identified and verified. These peptides are presented as SEQ ID NOs: 1-13.

### Bsi0359

Bsi0359 is an IgG type monoclonal antibody. The heavy chain variable amino acid sequence is represented in SEQ ID NO: 91 reproduced below (CDRs underlined).

EVQLVESGGGLVQPKGSLKLSCAASGFTFSTSAMNWVRQAPGKGLEWISR
IRSKTNNYATYYVDSVKDRFTIYRDDSQNMLYLQMNNLKTEDTAMYYCVR
EGDWGQGTLVTVSA

Peptides bound by Bsi0359 have been identified and verified. These peptides are presented as SEQ ID NOs: 39-42.

### D—Anti-C9 Antibodies

### Bsi0272

Bsi0272 is an IgG type monoclonal antibody. The heavy chain variable amino acid sequence is represented in SEQ ID NO: 86 reproduced below (CDRs underlined).

QVQLQQPGAELVRPGASVKLSCKASGYSFASYWMNWVKQRPGQGLEWIGM
IHPSDSGTSLDEKFKDKATLTVDKSSNTAYIQLNSPTSEDSAVYYCAREG
YD.PAWFAYWGQGTLVTVSA

The difference in biomarker level with Bsi0272 is represented FIG. 1, showing a very substantial difference between control and lung cancer.

Peptides bound by Bsi0272 have been identified and verified. These peptides are presented as SEQ ID NOs: 14-25.

### E—Anti-HPT Antibodies

### Bsi0033

Bsi0033 is an IgG type monoclonal antibody. The heavy chain variable amino acid sequence is represented in SEQ ID NO: 77 reproduced below (CDRs underlined).

EVQLQQSGADLVKPGASVKLSCTASGFNIKDTYMHWMKQRPEQGLEWIGR
IDPANGNSKYDPKFQGKATITADTSSNTAYLQLSSLTSEDTAVYFCTKSA
GVPFAYWGQGTLVTVSA

The difference in biomarker level with Bsi0033 is represented FIG. 1, showing a very substantial difference between control and lung cancer. Peptides bound by Bsi0033 have also been identified and verified. These peptides are presented as SEQ ID NOs: 36-38.

### Bsi0071

Bsi0071 is an IgG type monoclonal antibody. The heavy chain variable amino acid sequence is represented in SEQ ID NO: 81 reproduced below (CDRs underlined).

EVMLVESGGGLVKPGGSLKLSCAASEFTFSNYAMSWVRQTPEKRLEWVAT
ISSGGSFTYYPDNLKGRFTVSRDNAKDTLYLQMSSLRSEDTAIYYCARQS
LGYYFDSWGQGTTLTVSS

### F—Anti-CFH antibodies

Complement Factor H (NM_{—}000186) is a member of the Regulator of Complement Activation (RCA) gene cluster. The CFH protein contains twenty short consensus repeat (SCR) domains, is secreted into the bloodstream, and has an essential role in the regulation of complement activation, restricting this innate defense mechanism to microbial infections. Mutations in this gene have been associated with hemolytic-uremic syndrome (HUS) and chronic hypocomplementemic nephropathy. Complement factor H(CFH) is an inhibitor of the alternative complement pathway.

CFH detection, when combined with panels of the present invention, may further increase the reliability of the prediction.

Any anti-CFH antibody may be used to detect CFH. Specific examples of such antibodies include Bsi0077, Bsi0271, Bsi0885 and Bsi0893, as disclosed below.

### Bsi0077

Bsi0077 is an IgG type monoclonal antibody. The heavy chain variable amino acid sequence is represented in SEQ ID NO: 83 reproduced below (CDRs underlined).

EVQLQQSGPVLVKPGASVKISCKTSGYTFTEYTIHWMRQSHGKSLEWIGG
INPNKGNTNFNQKFKGKATLTVDKSSSTAYMELHSLPSEDSAVFYCARAN
WDVYAVDSWGQGTSVTVSS

Antigen binding was determined by sandwich reaction with BSI0271 and direct binding to purified natural CFH.

### Bsi0271

Bsi0271 is an anti-CFH IgG type monoclonal antibody which binds a peptide sequence selected from SEQ ID NOs: 43-56.

Additional anti-CFH antibodies include IgG Bsi0893 and Bsi0885, which bind a peptide sequence presented below, respectively.

| Ab | | Sequence | SEQ ID |
|---|---|---|---|
| Bsi0893 | CFH | CLPYPLCRGTAT | 75 |
| | | TECLPYPLCLYN | 76 |
| | | NETWSEFWRLHN | 78 |
| | | TAQDDRIFHMMH | 79 |
| | | RGTQDMEYFAMH | 80 |
| Bsi0885 | CFH | TVNPLLRLLSMG | 64 |
| | | NNPMLVLLSHGA | 65 |
| | | TVPQPQRLTQKS | 66 |
| | | AKYESLLRMLAA | 67 |
| | | GHESGYHISERA | 68 |
| | | GHEEHLWQILHF | 69 |
| | | GHEEQRNWSMEG | 70 |
| | | STYTDMLADLSA | 71 |
| | | SYESLLMKLARG | 72 |
| | | YRDSPGPRDKLL | 73 |
| | | GHEEREYQMMLQ | 74 |

In a specific assay format, CFH can be detected using the following combination of reagents:

| Capture reagent | Detection Ab | Target Protein |
|---|---|---|
| Bsi0271 | Bsi0885 | CFH |
| Bsi0893 | Bsi0885 | CFH |

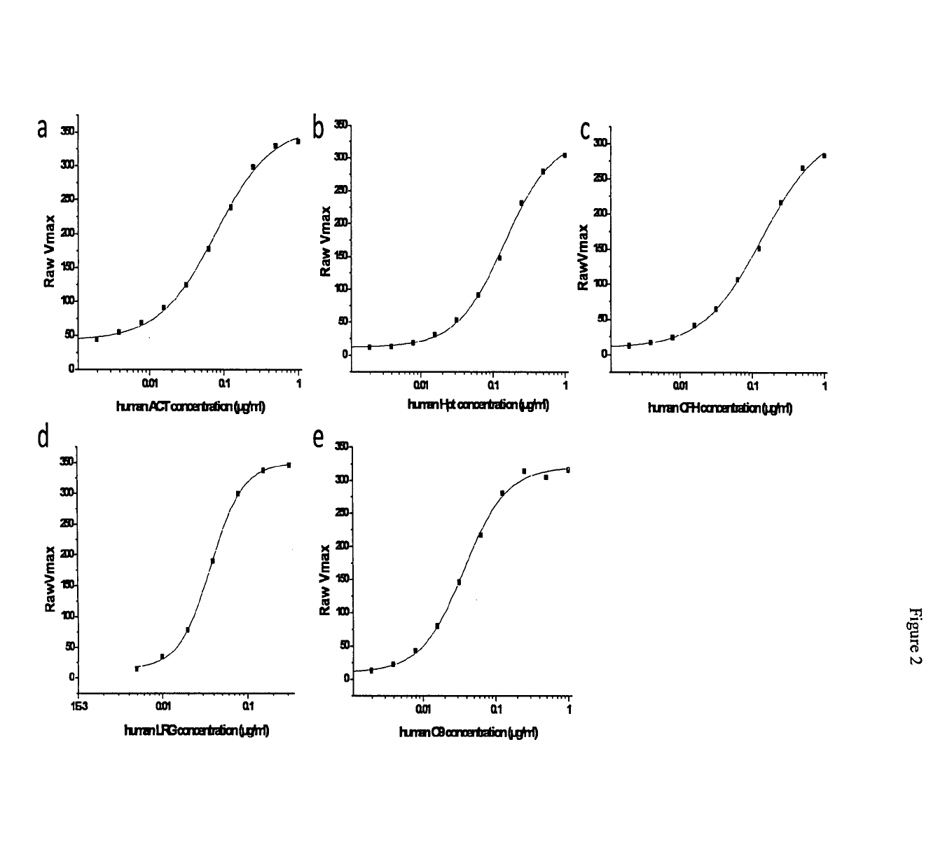

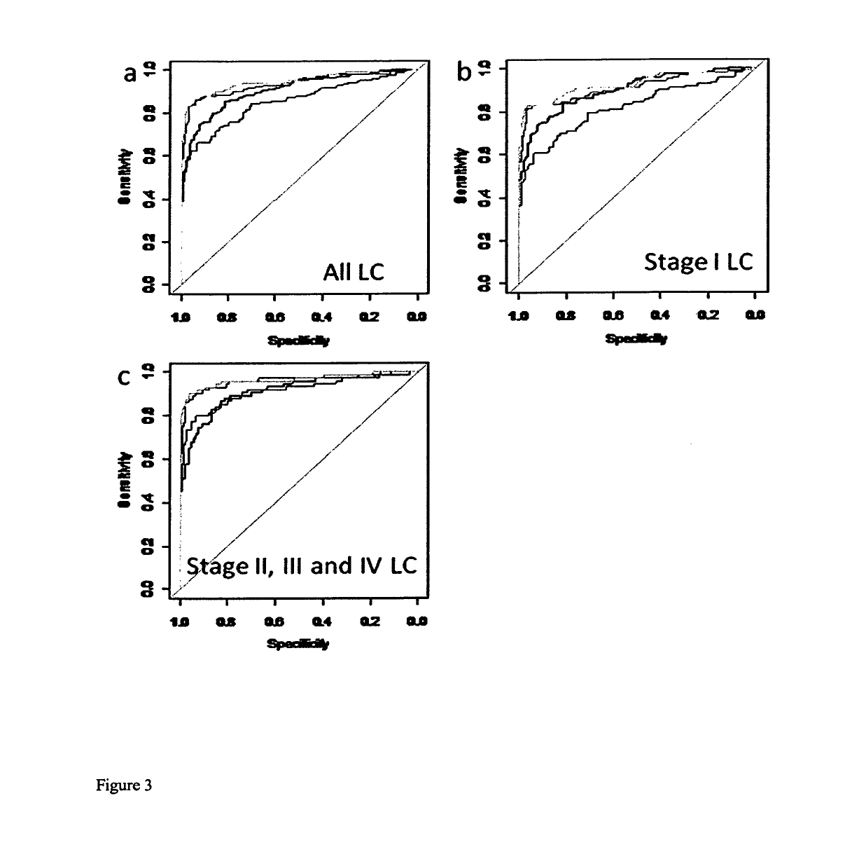

### G. Lung Cancer Biomarker Combinations (FIG. 1-3)

We have measured the concentration of the six biomarkers in plasma of lung cancer patients and healthy subjects using sandwich ELISA assays. Hpt, ACT, and LRG-1 proteins were measured using SW ELISA assays (FIG. 1-3) and cytokeratin-19 was measured using a commercially available kit CYFRA 21-1 EIA (Fujirebio). The discrimination capabilities of the individual biomarkers are quite good and their potential as diagnostics tools was determined from the area (AUC) under the receiver operating characteristics curves (ROC). The two biomarkers with the best performance as determined by the AUC were LRG1 and CYFRA21-1 having an AUC of 0.85 and 0.91 respectively, followed by ACT (AUC of 0.81), C9 (AUC of 0.80), and Hpt (AUC of 0.74) (Table below). The sensitivity at 95% specificity was also quite different among the different biomarkers and ranged from 67% for CYFRA 21-1 and 62% for LRG1 to 32% for HPT. Combining CYFRA21-1 with each one of the other biomarkers showed increased diagnostic performance and therefore added value. Using support vector machines, and ten fold cross validation of the results generated with the samples from the clinical cohort III (training set), we have determined multiple marker classifiers composed of combinations between the five biomarkers. An increase of more than 10% in the sensitivity at 95% specificity of the prediction was achieved when CYFRA21-1 was added to either a panel of three biomarkers (LRG-1, C9, ACT) or four biomarker panel (ACT+C9+LRG+Hpt). A remarkable performance was observed for a four-member panel with an AUC of 0.93 that could provide specificity of 95% with a sensitivity above 84%. In stages 1I and later, the panel reached 90% sensitivity.

TABLE
Performance of combined biomarkers

| | Se at | | | Se at | |
|---|---|---|---|---|---|
| | 95% Sp | AUC | | 95% Sp | AUC |
| Cyfra | 67.39 | 0.91 | | | |
| LRG | 62.26 | 0.85 | LRG + Cyfra | 83.25 | 0.93 |
| C9 | 43.87 | 0.80 | C9 + Cyfra | 76.85 | 0.91 |
| ACT | 60.85 | 0.81 | ACT + Cyfra | 81.28 | 0.92 |
| Hpt | 32.08 | 0.74 | Hpt + Cyfra | 69.46 | 0.91 |
| ACT + C9 + | 63.68 | 0.85 | ACT + C9 + LRG + | 84.24 | 0.93 |
| LRG | | | Cyfra | | |
| ACT + C9 + | 72.17 | 0.90 | ACT + C9 + LRG + | 84.24 | 0.94 |
| LRG + Hpt + | | | Hpt + CFH + Cyfra | | |
| CFH | | | | | |

Se: sensitivity; Sp: specificity

### Binding Peptides Recognized by Antibodies Suitable for Use in the Invention.

| | Peptide | Exemplary |
|---|---|---|
| SEQ ID NO: | Sequence | Antibody |
| 1 | NNSYLDEEGTWL | Bsi0358 |
| 2 | FPTHNPTSPLDM | |
| 3 | DLNDPPFFTSVS | |
| 4 | TTHWDHPFYDNT | |
| 5 | AWHPSSPLDYGF | |
| 6 | ANSVVYKPSSPL | |
| 7 | TSYNTDHPFHYP | |
| 8 | SPSAPSSPLDSM | |
| 9 | VRACVDCDQPFY | |
| 10 | IAPSSFLDERTD | |
| 11 | TRPHTDPPFWWA | |
| 12 | EPDPPFYTHLTA | |
| 13 | SQMKPSSHLDWD | |
| 14 | HTLPQFLEWHKR | Bsi0272 |
| 15 | GQVVFQDWLLVR | |
| 16 | HENANKIAPWVQ | |
| 17 | QLKIAPWVIESP | |
| 18 | TDTLDSFLGRSP | |
| 19 | KPPRQVVAPWVM | |
| 20 | IAQTLPAFLATR | |
| 21 | TETLHEFLDGRK | |
| 22 | KHLEHPTYHLWH | |
| 23 | SLNTIAPWILTA | |
| 24 | KATTHNIAWWVA | |
| 25 | HDNINMAWWVEW | |
| 26 | EWYVSTSLLPLP | Bsi0352 |
| 27 | HIYYISESLLPM | |
| 28 | HVEYEIIEQMIY | |
| 29 | SPFFYTLTRVPM | |
| 30 | SSYLYTTQFVPL | |
| 31 | TGWMISTTLIPT | |
| 32 | YKYSYTTELILI | |
| 33 | SYTYTTHLLPLA | |
| 34 | TMTQYYYSTTHY | |
| 35 | QFWTITTTLVPH | |
| 36 | GPKMDALSKAEN | Bsi0033 |
| 37 | GPRWLDALFMAE | |
| 38 | HIGAHDSFETKH | |
| 39 | NNSYLDEEGTWL | Bsi0359 |
| 40 | AMNTVVCYDEPC | |
| 41 | TNSPLDELGTHP | |
| 42 | STNSHEHWPMSP | |
| 43 | NHFSSLGILTAA | Bsi0271 |
| 44 | IPQPSILTTPML | |
| 45 | LRLPNTYENAKN | |
| 46 | GPLSPAHLSQRN | |
| 47 | FPLPFDPEMLPL | |
| 48 | FPLPPEYSMKKD | |
| 49 | FKIPEGNNVSVL | |
| 50 | FRLPPHDSSDHH | |
| 51 | FFLPHSATLPSH | |
| 52 | FILPHRNGYIDT | |
| 53 | LKLPNPDTAAPT | |
| 54 | NNNMGNILLITP | |
| 55 | VLLVPHHSHQYD | |
| 56 | YLKESTHVLLAA | |
| 57 | HDDWYISTSHTD | Bsi0351 |
| 58 | SYTYTTHLLPLA | |
| 59 | EWYVYEKLFPLP | |
| 60 | YKYSYTTELILI | |
| 61 | HQYYYTSSLIYT | |
| 62 | TAWHTTEELIML | |
| 63 | NGYIFTTQLIWA | |

Peptides Recognized by the Antibodies Used for the Sandwich Assays

| | | | |
|---|---|---|---|
| Bsi0186 | ACT | SVPWWTQSLLMS | SEQ82 |
| | | DIWSTMEHNKYN | SEQ84 |
| | | HINAIQYPLPHT | SEQ85 |

### REFERENCES

1. Stewart, B. W. & Kleihues, P. World cancer report. 1, 181-188 (2003).
2. Rivera, M. P., Detterbeck, F. & Mehta, A. C. Diagnosis of lung cancer: the guidelines. Chest 123, 129S-136S (2003).
3. Kulpa, J., Wojcik, E., Reinfuss, M. & Kolodziejski, L. Carcinoembryonic antigen, squamous cell carcinoma antigen, CYFRA 21-1, and neuron-specific enolase in squamous cell lung cancer patients. Clin Chem 48, 1931-1937 (2002).
4. Zolg, W. The proteomic search for diagnostic biomarkers: lost in translation? Mol Cell Proteomics 5, 1720-1726 (2006).
5. Beane, J., Spira, A. & Lenburg, M. E. Clinical impact of high-throughput gene expression studies in lung cancer. J Thorac Oncol 4, 109-118 (2009).
6. Rifai, N., Gillette, M. A. & Carr, S. A. Protein biomarker discovery and validation: the long and uncertain path to clinical utility. Nat Biotechnol 24, 971-983 (2006).
7. Gao, W. M. et al. Distinctive serum protein profiles involving abundant proteins in lung cancer patients based upon antibody microarray analysis. BMC Cancer 5, 110 (2005).
8. Knezevic, V. et al. Proteomic profiling of the cancer microenvironment by antibody arrays. Proteomics 1, 1271-1278 (2001).
9. Maneewatch, S. et al. Human single-chain antibodies that neutralize homologous and heterologous strains and clades of influenza A virus subtype H5N1. Antivir Ther 14, 221-230 (2009).
10. Houimel, M. & Dellagi, K. Isolation and characterization of human neutralizing antibodies to rabies virus derived from a recombinant immune antibody library. J Virol Methods (2009).
11. Uhlen, M. et al. A human protein atlas for normal and cancer tissues based on antibody proteomics. Mol Cell Proteomics 4, 1920-1932 (2005).
12. Blow, N. Antibodies: The generation game. Nature 447, 741-744 (2007).
13. Csanky, E. et al. Monoclonal antibody proteomics: discovery and prevalidation of chronic obstructive pulmonary disease biomarkers in a single step. Electrophoresis 28, 4401-4406 (2007).
14. Peracaula, R., Barrabes, S., Sarrats, A., Rudd, P. M. & de Llorens, R. Altered glycosylation in tumours focused to cancer diagnosis. Dis Markers 25, 207-218 (2008).
15. Kullolli, M., Hancock, W. S. & Hincapie, M. Preparation of a high-performance multi-lectin affinity chromatography (HP-M-LAC) adsorbent for the analysis of human plasma glycoproteins. J Sep Sci 31, 2733-2739 (2008).
16. Kohler, G. & Milstein, C. Continuous cultures of fused cells secreting antibody of predefined specificity. Nature 256, 495-497 (1975).
17. Kendziorski, C., Irizarry, R. A., Chen, K. S., Haag, J. D. & Gould, M. N. On the utility of pooling biological samples in microarray experiments. Proc Natl Acad Sci USA 102, 4252-4257 (2005).
18. Page, M. & Thorpe, R. Hybridoma Production. The Protein Protocols Handbook, 773-775 (2002).
19. Zhang, J. H., Chung, T. D. & Oldenburg, K. R. A Simple Statistical Parameter for Use in Evaluation and Validation of High Throughput Screening Assays. J Biomol Screen 4, 67-73 (1999).
20. Shapiro, S. S. a. W., M. B. Biometrika 52, 591-611 (1965).

1-23. (canceled)

24. A method for the detection, diagnosis, staging or management of lung cancer in a patient, the method comprising the combined analysis of a CYFRA protein and at least a second protein selected from LRG-1, ACT, HPT, and C9, in a sample from the subject, said combined analysis providing an indication of the presence, stage or progression of lung cancer in the patient.

25. The method of claim 24, comprising the combined analysis of a CYFRA protein and a LRG-1 protein.

26. The method of claim 24, comprising the combined analysis of a CYFRA protein and an ACT protein.

27. The method of claim 24, comprising the combined analysis of a CYFRA protein and a HPT protein.

28. The method of claim 24, comprising the combined analysis of a CYFRA protein and a C9 protein.

29. The method of claim 24, comprising the combined analysis of a CYFRA protein, a LRG-1 protein, an ACT protein, and a C9 protein.

30. The method of claim 24, wherein the sample is a blood sample.

31. The method of claim 24, wherein analysing said proteins comprise contacting the sample, or an aliquot thereof, with a with a first specific binding reagent that binds a CYFRA protein and with at least a second specific reagent that binds a second protein selected from LRG-1, ACT, HPT, and C9, and determining the presence or amount of protein bound to said binding reagents.

32. The method of claim 31, wherein said contacting with the at least 2 binding reagents is performed simultaneously or sequentially, on the same sample or on different aliquots of a same sample.

33. The method of claim 31, wherein the binding reagents are antibodies, fragments or derivatives thereof.

34. The method of claim 24, wherein lung cancer is Stage I or II lung cancer.

35. The method of claim 24, wherein lung cancer is Stage Ia or IIa lung cancer.

36. The method of claim 33, wherein the binding reagent is an anti-LRG-1 antibody, fragment or derivative thereof which binds human LRG-1 and which binds a peptide having a sequence selected from SEQ ID NOs: 26-35 and 57-63.

37. The method of claim 33, wherein the binding reagent is an anti-C9 antibody, fragment or derivative thereof which binds human C9 and which binds a peptide having a sequence selected from SEQ ID NOs: 14-25.

38. The method of claim 33, wherein the binding reagent is an anti-HPT antibody, fragment or derivative thereof which binds human HPT or HPRT and which binds a peptide having a sequence selected from SEQ ID NOs: 36-38.

39. The method of claim 33, wherein the binding reagent is an anti-CYFRA antibody, fragment or derivative thereof which binds human cytokeratin 19 or a fragment thereof, preferably CYFRA21-1 fragment, or protein or peptide comprising a sequence selected from SEQ ID NOs: 93-96.

40. The method of claim 33, wherein the anti-ACT antibody, fragment or derivative thereof is an antibody, fragment or derivative thereof which binds human ACT and which binds a peptide having a sequence selected from SEQ ID NOs: 1-13 and 39-42.

41. A device comprising at least one antibody, fragment or derivative thereof, that binds a CYFRA protein and at least one antibody, fragment or derivative thereof, that binds a protein selected from Leucine-Rich alpha-2 glycoprotein, haptoglobin, C9, or Alpha 1 Antichymotrypsin, immobilized on a support.

42. The device of claim 41, wherein the support is a membrane, a slide, a microarray, a chip or a microbead.

43. A kit comprising a device of claim 41 and a reagent to perform or detect (or quantify) an immune reaction, particularly an antibody-antigen complex.
